(19) United States
(12) Patent Application Publication
(10) Pub. No.: US 2005/0090992 A1
(43) Pub. Date: Apr. 28, 2005

(54) Using multiple perturbations to elucidate connectivity in network systems

(76) Inventors: Joseph Lehar, Lexington, MA (US); Raymond A. Molnar, Boston, MA (US); Curtis T. Keith, Boston, MA (US); Grant Zimmermann, Somerville, MA (US)

Correspondence Address: BROMBERG & SUNSTEIN LLP, 125 SUMMER STREET, BOSTON, MA, 02110-1618, US

(21) Appl. No.: 10/951,275

(22) Filed: Sep. 27, 2004

Related U.S. Application Data
(60) Provisional application No. 60/506,401, filed on Sep. 26, 2003.

(51) Int. Cl. G06F 17/60; G06G 7/48; G06G 7/58; G06F 19/00; G01N 33/48; G01N 33/50

(52) U.S. Cl. 702/19; 703/11; 705/2

(57) ABSTRACT

One embodiment of the invention is a method of analyzing the effects of combined perturbers of a network system by means of a set of model predictions for various network configurations, using a set of phenomenologically-based combination surface models. This method can be used to predict the effects of combined perturbers of known networks, for example, providing mechanistic validation for therapeutic compounds. Alternatively, this method can provide constraints for constructing connectivity models from observed combination effects on networks of unknown structure, thus, for example, providing the required understanding to identify novel targets for therapeutic compounds.

### CROSS REFERENCE TO RELATED APPLICATIONS

This application gains priority from provisional application Ser. No. 60/506,401 filed on Sep. 26, 2003 and incorporated herein by reference.

### TECHNICAL FIELD

The present invention relates to methods of identifying relationships in network systems, especially systems utilizing chemical, biochemical, and biological mechanisms.

### BACKGROUND ART

Understanding connectivity in network systems continues to be challenging in many fields. For example, as the science of biology advances, increasing attention is being focused on the problem of understanding biological systems in their entirety. In “systems biology”, large amounts of data are being collected to record the state of the many components in specific organisms or organic pathways, which are then used to constrain a global network model of the system.

In a typical enterprise, a research group will select a model system (a cancer cell culture, for example), and monitor multiple variables under a few critical conditions (e.g., starvation, temperature variations, or treatment by various antineoplastics). The collected data are then used as constraints, eliminating many of the possible network models for the system.

Due to the complexity of biological systems, such studies require immense amounts of data. Current sources of data for systems biology include gene expression profiling using cDNA microarrays, protein expression profiling using two-dimensional gels, and metabolite profiling using mass spectrum analyzers. Even in the presence of all these data, most efforts at systems biology are largely under-constrained, in the sense that the various network models that fit the data still encompass a wide range of biological variation. For this reason, new sources of high-content biological constraints are key.

Perturbations by biologically active compounds provide some of the most informative data for systems biology. They permit not just the observation of conditions, but also the manipulation of systems, allowing predictive studies which would not be possible with traditional expression profiling and mass spectroscopy.

Multiple perturbing compounds can exponentially increase the available constraints on a system. When multiple perturbers act upon a system, they may have no combined effect or they may act together in a synergistic or antagonistic manner. The most standard reference for synergy is the Loewe additivity model [Loewe 1928, Ergeb. Physiol. 27:47], which provides the basis for the Combination Index [Chou & Talalay 1984, Adv Enzyme Regul. 22:27-55], which is the most widely applied method of synergy determination. These references, and all others identified in this application, are hereby incorporated herein by reference.

The study of compound combinations as constraints to biological systems was pioneered by the work of Robert Jackson [e.g., Jackson & Harrap 1973, Arch. Biochem & Biophys 158:827-841; Jackson 1993, Cancer Res 53:3998-4003]. In extensive computational simulations, he found that the behaviour of multiply-perturbed enzymatic pathways was very dependent on the relative position of each perturber's target in the pathway. Recent work on the folate nucleotide synthesis pathway produced an experimental example of connected targets leading to combination effects [Faessel et al. 1998, Cancer Res 58:3036]. Recently, in an experiment involving ten mutant strains of yeast treated with all combinations of 24 biologically active compounds, Haggerty et al. [2003, J. Am. Chem. Soc. 125:10543-5] were able to show that the various strains of yeast produce different compound combination profiles.

All of these foregoing studies, however, could provide only limited information from each combination of perturbers. Jackson consistently found that it was difficult to predict the level of synergy or antagonism for enzymatic networks, since the resulting combination index was highly sensitive to the specific values of the various cytokinetic parameters. This prevented studies like Faessel et al. from testing predictive combination models of the folate pathway. The chemical genomic screening experiment by Haggerty et al. was likewise limited to building a network of chemical associations without addressing the underlying structure of the biological network. Jackson's analysis was also limited to a comparison with Loewe additivity only.

### SUMMARY OF THE INVENTION

In a first embodiment of the invention, a method of elucidating connectivity in a network system that has been subjected to a plurality of agents, the agents having an interaction in the system, is provided. The method includes the steps of providing a set of interaction models for describing an interaction of agents in the system; selecting an interaction model from the set that best models the interaction of agents in the system; and relating the selected model to connectivity of the network. The network system may include at least one of a chemical system, biochemical system, and biological system. The plurality of agents may include at least one composition. The composition may include a pharmaceutically active composition; an entity approved by a governmental regulatory agency for administration to a patient; or an entity having at least one of an established safety profile, a recognized pharmacology profile, and a recognized toxicity profile. Selecting the interaction model may be based on a least squares method.

Interaction models that may be used with embodiments of the invention described herein include: (1) the Loewe additivity model; (2) an Independence model that encompasses parallelism, branching, Bliss, and bypass models; (3) the Greco synergism model, and (4) a Potentiation model.

The Loewe additivity model applies to cases where both components of the combination affect the same location in the pathway in a similar manner. The response to such combinations follows the constraint that:

$$\frac{{C}_{X}}{{EC}_{X}} + \frac{{C}_{Y}}{{EC}_{Y}} = 1$$

where C_{X}, C_{Y} are the concentrations of the two agents for a particular combination treatment, and EC_{X}, EC_{Y} are the “effective concentrations” of the single agents (the single agent concentrations that can produce the same level of effect as at the specified combination).

The Independence model applies to cases where the targets are independent locations in the pathway, wherein the combined inhibition I at concentrations C_{X}, C_{Y} produced is:
I=X+Y−XYγ
where X,Y are the inhibitions of the single perturbers at C_{X} and C_{Y}, respectively. The interaction parameter, gamma (γ), describes the degree to which the single agents interact to produce a combination effect. Gamma takes on different values for specific placements of targets, some of which are shown in FIG. 2. The special case of γ=1 corresponds to Bliss Independence [Bliss, 1939, Ann. Appl. Biol. 26:585-115], and is the expected result when the two perturbers are placed serially in the network. Other special cases occur when the targets are at different arrangements, as described later in this application.

The Greco Synergism model [Greco, W R, Park, H S, Rustum, Y M, 1990, Cancer Res. 50: 5318-5327] may be applied to cases where the targets are placed to produce one of the independence models as described above, but when the inhibitions were calculated after several rounds of exponential expansion (for example, generations of proliferation). Very much like Loewe additivity, Greco synergism obeys the constraint:

$$\frac{{C}_{X}}{{EC}_{X}} + \frac{{C}_{Y}}{{EC}_{Y}} + α ( \frac{{C}_{X}}{{EC}_{X}} \frac{{C}_{Y}}{{EC}_{Y}} ) = 1$$

permitting a smooth transition from highest single agent effect (α=−1) through Loewe additivity (α=0) to very strong potency shifting (as a grows to very large values) [Greco et al., 1990]. As with Loewe additivity, the combination response value for each C_{X}, C_{Y} pair must be determined using numerical root-finding.

Finally, a Potentiation model may be applied to cases where the Y compound directly increases or decreases the X compound's ability to inhibit the biological process. The inhibition for a potentiated model is:
I=X(C′_{X})
where C′_{X} is C_{X}(1+C_{Y}/C₀)^{π}.

Here, C₀ is the threshold Y concentration at which potentiation becomes important, and pi (π) is the potentiation index governing the degree of synergism produced.

In a related embodiment of the invention, a method of identifying an interacting agent having an interaction with a network system is revealed. The method includes the steps of the first embodiment and further includes the step of identifying the interacting agent having the interaction in the network system based on the connectivity of the network. The interacting agent may or may not be one of the plurality of agents, may be at least part of a pharmaceutically active composition, and may include an entity approved by a governmental regulatory agency for administration to a patient. A pharmaceutically active composition may be produced by this related embodiment, where the composition includes the interacting agent and another agent identified based on the interaction between another agent and the interacting agent in the network. The related embodiment may also provide a method of using a pharmaceutically active composition to produce an interaction in an organism, the method including the steps of identifying an interacting agent; combining the interacting agent with another agent, identified based on the interaction between the another agent and the interacting agent in the network system, to produce the pharmaceutically active composition; and administering the pharmaceutically active composition to the organism, the organism having the network system.

In another related embodiment of the invention, a method of identifying an interacting agent with an interaction in a particular network system is provided. The method further includes the steps of repeating the steps of the first embodiment of the invention for each of a plurality of network systems; and identifying the interacting agent with the interaction in the particular network system based on the connectivity of at least one of the plurality of network systems. The interacting agent may or may not be one of the plurality of agents, may be at least part of a pharmaceutically active composition, and may include an entity approved by a governmental regulatory agency for administration to a patient. The particular network system may or may not be one of the plurality of network systems. A pharmaceutically active composition may be produced by this embodiment, where the composition includes the interacting agent and another agent identified based on the interaction between another agent and the interacting agent in the particular network. The another related embodiment may also provide a method of using a pharmaceutically active composition to produce an interaction in an organism, the method including the steps of identifying the interacting agent; combining the interacting agent with another agent, identified based on the interaction between the another agent and the interacting agent in the particular network system, to produce the pharmaceutically active composition; and administering the pharmaceutically active composition to the organism, the organism having the particular network system.

Still another related embodiment of the invention relates to a method of elucidating a potential mechanism of interaction of a particular composition according, wherein the plurality of agents includes at least one composition. Using the steps of the first embodiment, this method further includes the step of identifying the potential mechanism of interaction of the particular composition in a particular system based on the connectivity of the network. The particular composition may or may not be the at least one composition, and may include an entity approved by a governmental regulatory agency for administration to a patient.

In yet another related embodiment of the invention, a method of preparing a high throughput screen is revealed. The method includes the steps of the first embodiment and further includes the step of preparing a high throughput screen based on the connectivity of the network.

In a second embodiment of the invention, a method of elucidating connectivity in a network system that has been subjected to a plurality of agents is shown. The method includes the steps of providing a set of interaction models for describing an interaction of agents in the system; determining an interaction of at least one of the plurality of agents in the system; selecting an interaction model from the set that best models the interaction of agents; and relating the selected model to connectivity of the network. Determining the interaction may include using a high throughput screening method. The embodiment may also include the use of at least three agents in the plurality of agents, and further include the steps of selecting at least one more interaction models from the set, each interaction model best models a particular interaction of agents in the system; and relating each selected model to the connectivity of the network.

In a third embodiment of the invention, a method of producing an interaction model to describe an interaction of agents in a network system for elucidating connectivity in the system is shown. The method includes the steps of simulating interaction of agents in the system to produce a response surface; and producing the interaction model based on the response surface.

Other embodiments of the invention utilize a computer program product for use on a computer system to practice the methods discussed herein.

### BRIEF DESCRIPTION OF THE DRAWINGS

The foregoing features of the invention will be more readily understood by reference to the following detailed description, taken with reference to the accompanying drawings, in which:

FIG. 1 depicts a combined inhibition surface as a function of the concentration of two agents, and the corresponding difference surface between the combined inhibition and the highest single agent inhibition of the two agents acting independently;

FIG. 2 depicts a simple branched connectivity diagram of a network system;

FIG. 3 depicts the combined inhibition surfaces, as in FIG. 1, produced by various arrangements of target pairings within the network diagram of FIG. 2; and

FIG. 4 depicts the results of a yeast pathway experiment, documenting which interaction models best fit a particular measured response surface corresponding to a particular pair of interacting agents.

### DETAILED DESCRIPTION OF SPECIFIC EMBODIMENTS

Definitions. As used in this description and the accompanying claims, the following terms shall have the meanings indicated, unless the context otherwise requires:

An “agent” is any composition or physical or chemical quantity potentially capable of interacting with another agents or part of a network system. Non-limiting examples of agents include chemical compounds, biological entities, heat, radiation, electrical fields or forces, and magnetic fields or forces.

A “composition” is a set of one or more entities that constitute a discrete sample. Each composition may include the same or a different set of entities, compared with any other composition. The absolute amount and concentration of a particular entity within a composition may match or differ from the absolute amount or concentration of the entity in any other composition. Thus two compositions can be the same, though they differ in the concentration or quantity of one or more entities.

“Effective concentrations” EC_{X} and EC_{Y} of single agents as used herein means the single agent concentrations that are required to produce a specified effectiveness level.

“Connectivity of a network system” refers to a relationship between a plurality of differing portions of a network system.

An “entity” is a component of a composition. Some non-limiting examples of specific entities include a chemical substance; a drug; a biological moiety; and a substrate capable of holding a chemical substance, drug, or biological moiety (e.g. small polymeric particles with an absorbed layer of an organic molecule). An entity may be a component of an assay for analysis of a compound, or may be the compound itself or a component of the compound.

An “interaction” refers to any type of effect between a plurality of agents or between at least one agent and a system. The term “interaction” does not presuppose a particular outcome. For example, in the case of an interaction of agents, the outcome may be synergetic, antagonistic, additive, a nullity, etc.

A “network system” refers to the potential pathways and mechanisms by which one or more end points may be related to one or more starting points. The points may be defined by any set of environmental, physical, or chemical variables. Network systems, for which embodiments of the invention may be applied, include environmental, chemical, biological, and biochemical systems. Network systems may also involve pathways and mechanisms that hybridize two or more types of systems (e.g. a network system may embed a chemical and biological system). The term “network system” is not meant to limit the degree of complexity or simplicity between any starting point and any ending point.

In a specific embodiment of the invention, simulations of pairwise combinations of agents, herein also known as perturbers, in an enzymatic pathway are used to produce four interaction models of combination surface models, each associated with a specific relationship between the targets of the perturbers of the network system. A surface refers to the set of responses, or end points (measured, calculated, or simulated), as a result of the interaction of agents in a system. In addition to Loewe additivity, three novel models are produced, which together may describe the combination surfaces for inhibitors that target different sites in the network. It is worth noting that Bliss independence, an early alternative synergy model based on probabilistic arguments [Bliss, 1939, Ann. Appl. Biol. 26:585], is a special case of the Independence model.

Some embodiments of the invention utilize sets of interaction models to describe a particular response surface generated by the presence of a plurality of agents acting on a network system. As a result, some embodiments of the invention may be used to show that surfaces generated by the same inhibited network have the same topology, despite their containing regions of both Loewe synergy and antagonism. The models, thus, may be related to the connectivity of the network system to which the model is applied. Knowledge of the connectivity of the network system may be used to identify interacting agents having a desired, or undesired, interaction in the network system or other similar network systems as understood by those skilled in the art. Moreover, the identified interacting agents may be agents that were used in the creating the response surface, or may be agents that were not present in the response surface but are identified on the basis of analogous physical, chemical, or other features with agents that are used in the response surface (the analogous features being those that would be recognized by persons skilled in the relevant art). Such identified interacting agents may be compositions. Non-limiting examples of compositions include pharmaceutically active compositions; compositions including an entity approved by a governmental regulatory agency for administration to a patient; and compositions having at least one of an established safety profile, a recognized pharmacology profile, and a recognized toxicity profile. An interacting agent may also be a component of some composition.

Several embodiments of the present invention are directed toward biological network systems and biochemical network systems. Those skilled in the relevant art, however, will readily understand that methods relating to using interaction models to describe the interaction of agents in a network system may be applied to network systems beyond those described in particular embodiments herein. For example, network systems in some chemical reaction schemes may have similar structure and connectivity. These chemical reaction schemes may be found in the context of understanding the chemical kinetics of molecular interaction in a particular reaction scheme, or may involve more heterogeneous systems that include transport phenomena with the chemical kinetics (e.g., industrial catalytic processes). Other examples of network systems that may have similar structures or connectivity are in the fields of chemical, electrical, or mechanical automatic process control; and environmental systems (e.g., ecological or climate systems, both local and global). Thus embodiments of the invention may be used in any network system having a connectivity suitable for the methods described herein, as perturbed by the presence of particular agents.

Some embodiments of the invention described herein utilize a description of the effect or response surfaces produced by the multiple perturbers. As known to those skilled in the art, many types of response surfaces may be created using different types of variables. In particular embodiments of the invention, if a biological system has a measured end point (final condition) which is fully inhibited by the presence of a compound at high concentrations, we would expect to see 0% inhibition when no compound is present, and 100% inhibition when its concentration is high, where inhibition I is defined by the equation:
I=untreated−treated/untreated
where untreated is the measured end point when the biological system is not exposed to a perturber; and treated is the measured end point when the biological system is exposed to a perturber.

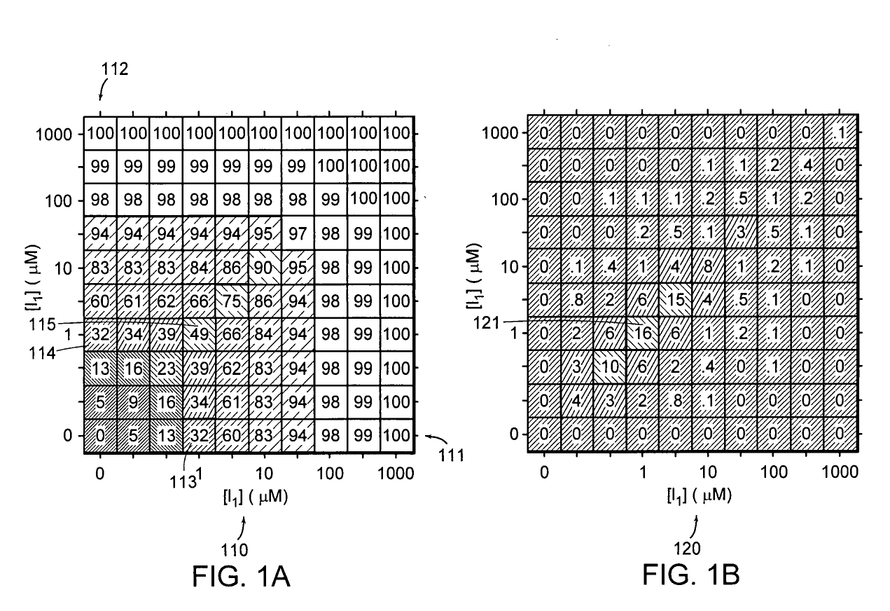

When two perturbers are applied to the system, the combination response surface is defined by the observed inhibition as a function of both concentrations of the perturbers (herein also called inhibitors in the context of inhibition surfaces). For example, FIG. 1 shows a combined response surface for a biological system in the form of an inhibition surface. The left hand panel 110 shows the inhibition as a function of both inhibitor concentrations. The values increase from 0 at the origin to 100% as each compound reaches its maximum concentration. The individual dose response curves can be seen along the bottom and left axes 111, 112. The right hand panel 120 shows the difference between the observed inhibition surface and the highest single agent inhibition at the same concentration. So, at single agent concentrations C_{X}=C_{Y}=1, both single agents have an inhibition of 32% as indicated by the corresponding surface values 113, 114, while the combination is inhibited at 49% as indicated by the corresponding surface value 115. The combination value is 16% higher than the highest single agent value of 32%, the difference value indicated by the corresponding surface value 121 in the right hand panel 120. Inhibition surfaces may be obtained from computer simulations, or actual measured values from experimentation.

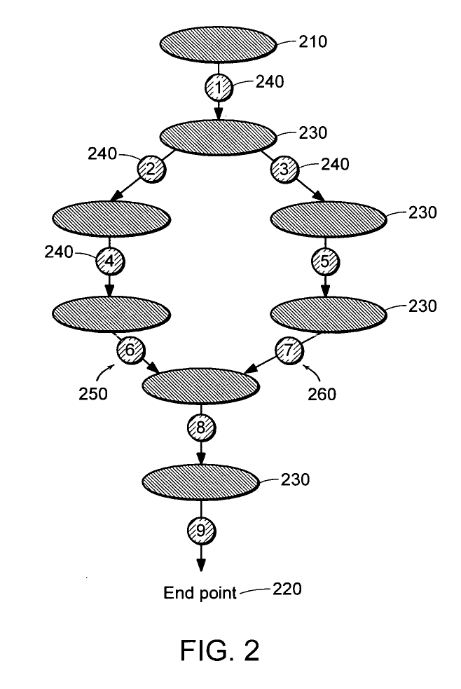

Inhibition surfaces may be used to provide information on the connectivity of a network system. One example of a network system with a simple branched network is depicted by the connectivity diagram in FIG. 2. A supplied input compound 210 is converted into an end-point product. 220 via many intermediate stages 230 as mapped out by the pathway. Each stage of the reaction is mediated by catalysts 240. The reaction can proceed along either of two pathways 250, 260.

In an embodiment of the invention, an enzymatic network with the structure of FIG. 2 is simulated using the techniques of Jackson [1993]. The simulations produced a wide variety of combination response surfaces. Although the precise shape of each surface and the synergy (as measured by the Combination Index) both varied substantially in response to changes in the kinematic parameters, the topology (or type) of each surface remained the same for a particular configuration of inhibitors in the network.

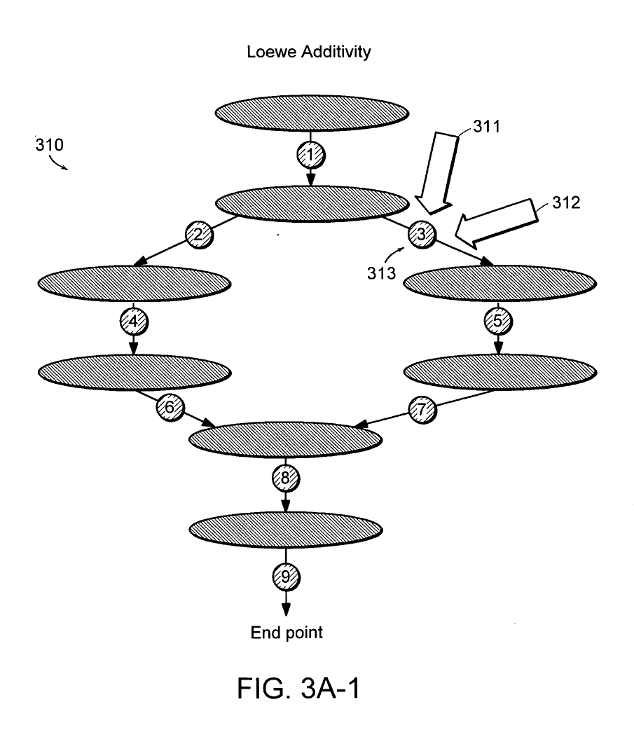

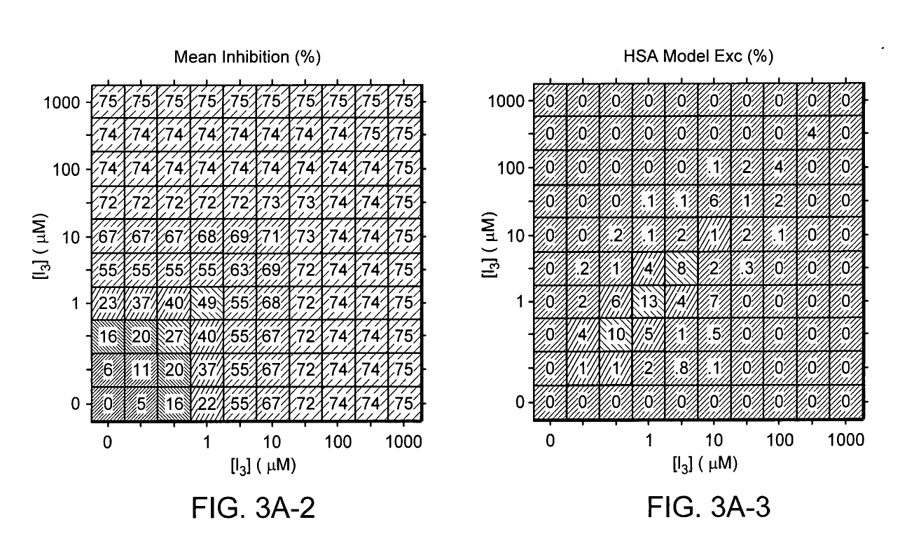

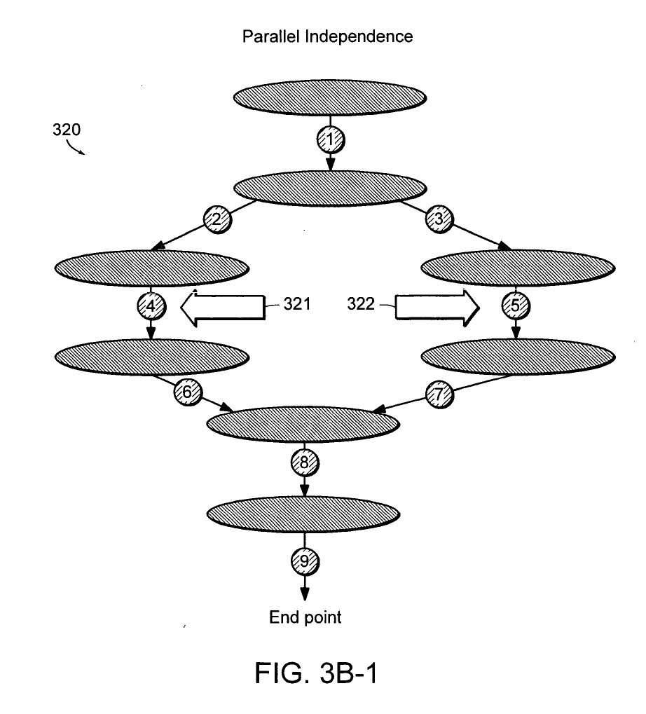

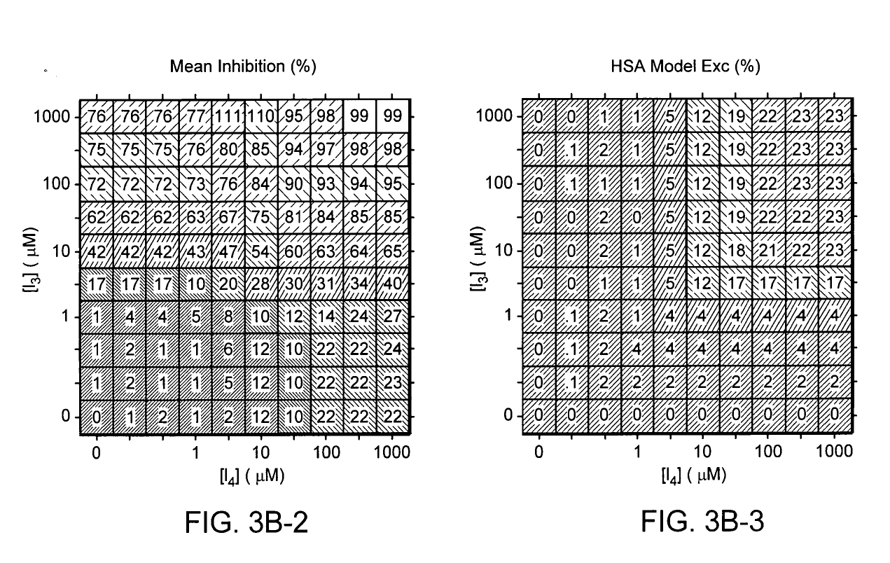

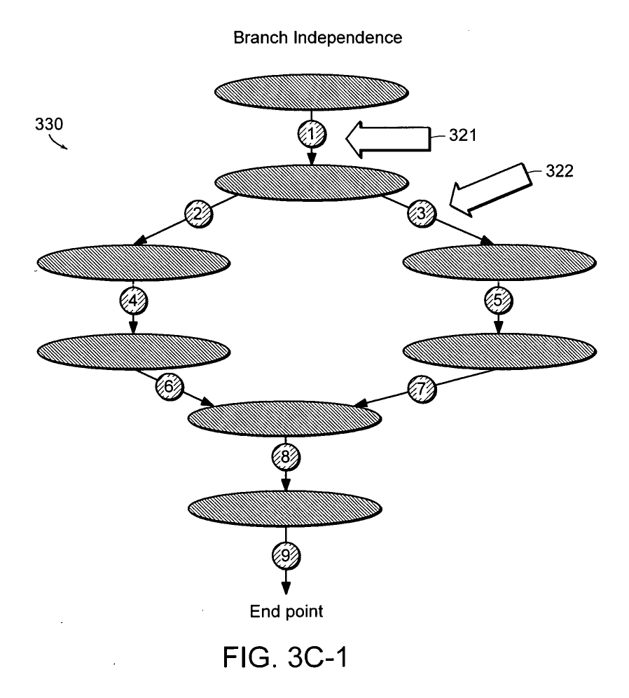

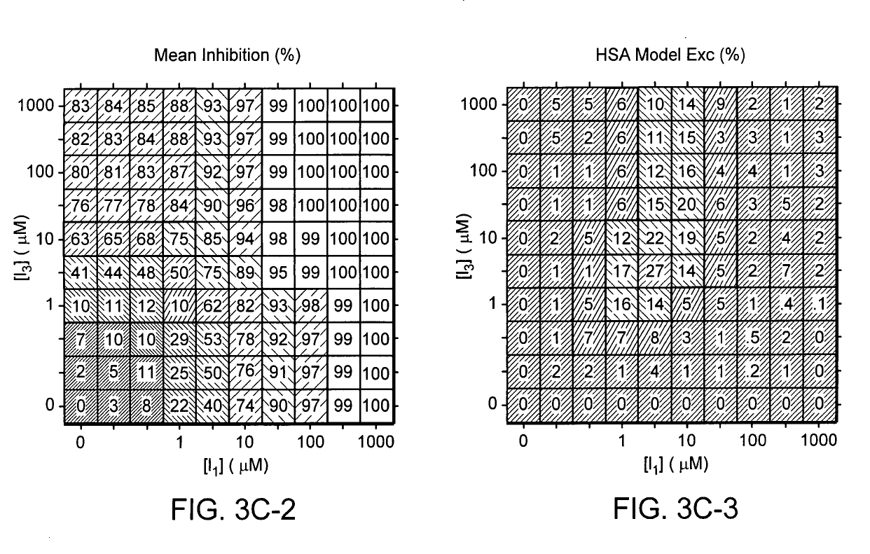

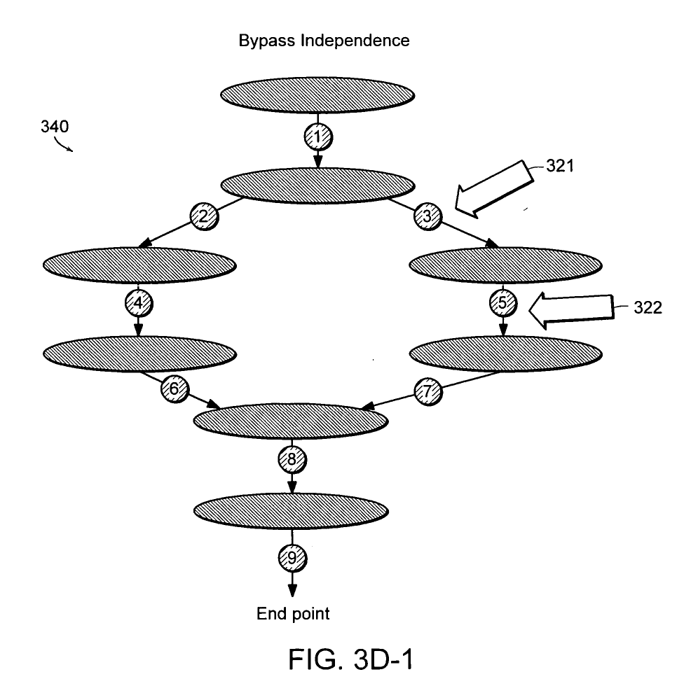

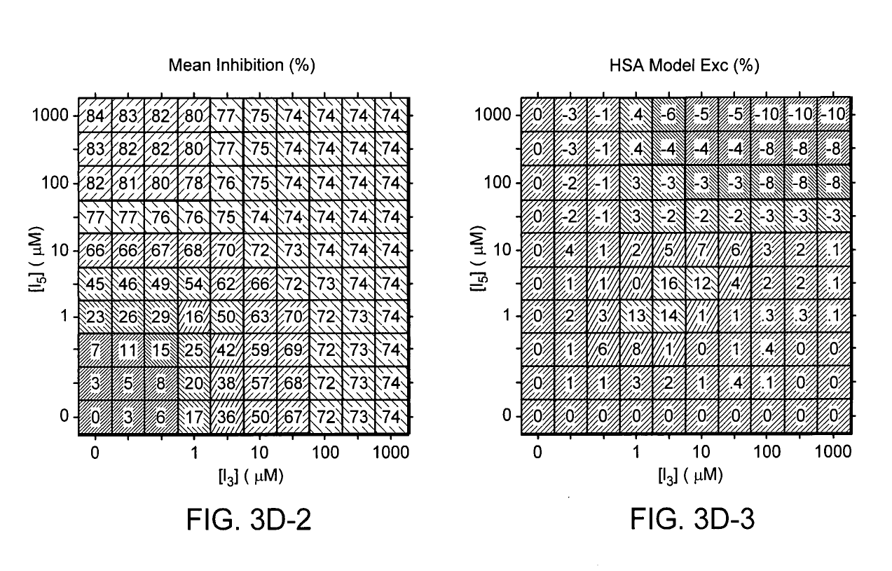

The combination response surfaces produced for pairs of inhibitors, in an embodiment of the invention, may be modeled by one of four phenomenologically-based models: the Loewe Additivity model; the Independence model; the Greco synergism model; and the Potentiation model. Some of these models are known to those in the art, but the general Independence model and the Potentiation model are each embodiments of the present invention. The application of all of these models to the network contexts described herein, however, are each novel embodiments of this invention. Graphical representations of the network connectivity of some of these models, along with corresponding representative inhibition surfaces and difference value surfaces from the highest single agent, are depicted by the embodiment shown in FIG. 3. The novel models exhibit limited potency shift.

Loewe Additivity: This model may be applied when both perturbers 311, 312 target the same point 313 in the biological network 310. This model applies for any location in the network provided only that both perturbers target the same site.

Loewe additivity obeys the following mathematical constraint:

$$\frac{{C}_{X}}{{EC}_{X}} + \frac{{C}_{Y}}{{EC}_{Y}} = 1$$

where C_{X} and C_{Y} are the concentrations of the perturber, while EC_{X} and EC_{Y} are their effective concentrations at the same effectiveness level. Loewe additivity has the property that the combined inhibition level never exceeds that of the perturbers separately, but that substantial potency increases (similar effect at lower concentrations) can occur. As there is no closed-form solution to this constraint equation, the effect level satisfying this constraint must be determined at each C_{X}, C_{Y} using numerical root-finding techniques. The Loewe Additivity model as specified is not limited to inhibition measurements, and applies in general to any quantitative measure of effect.

Independence Model: This model applies to cases where the targets are independent locations in the pathway. The combined inhibition I produced is:
I=X+Y−XYγ
where X,Y are in the inhibitions of the single perturbers at C_{X} and C_{Y}, respectively. The interaction parameter, gamma (y), describes the degree to which the single agents interact to produce a combination effect. Gamma takes on different values for specific placements of targets, some of which are shown in FIG. 3 (e.g. networks 320, 330 and 340). Examples for values of gamma include:
γ=1 (Bliss Independence) Applies for serial targets;
γ=(X_{∞}+Y_{∞}−1)/(X_{∞}Y_{∞}) “Parallel independence (network 320 of FIG. 3)”, corresponds to the Parallel placement of inhibitors 321, 322;
γ=1/max(X_{∞},Y_{∞}) “Branch independence (network 330 of FIG. 3)”, applies when the inhibitors 321, 322 straddle a branch point;
γ=1/min(X_{∞}, Y_{∞}) “Bypass independence (network 340 of FIG. 3)”, applies when both inhibitors 321, 322 are on a bypassed branch;
γ=0 “Bypassed parallelism”, applies to parallel inhibitors that are both bypassed; and
γ=(X_{∞}+Y_{∞})/(X_{∞}Y_{∞}) “Antagonistic independence” corresponds to complete antagonism;
where X_{∞} and Y_{∞} are the limiting effect levels at very high concentrations of the single agents. Although the Independence model as specified herein applies only to effects measured as inhibitions, the model can be easily modified to be used in the context of other effect measures, for example a simple ratio of the treated to the untreated experimental response.

Greco Synergism: This model (Greco W R, Park H S, Rustum Y M, 1990, Cancer Res. 50: 5318-5327) may be applied to cases where the targets are placed to produce one of the independence models as described above (Parallelism, Bypass, Branch, Bliss) but when the inhibitions were calculated after several rounds of exponential expansion (for example, generations of proliferation). Greco synergism obeys the constraint:

$$\frac{{C}_{X}}{{EC}_{X}} + \frac{{C}_{Y}}{{EC}_{Y}} + α ( \frac{{C}_{X}}{{EC}_{X}} \frac{{C}_{Y}}{{EC}_{Y}} ) = 1$$

Greco synergy extends Loewe additivity by permitting a smooth transition from highest single agent effect (α=−1) through Loewe additivity (α=0) to very strong potency shifting (as a grows to very large values) [Greco et al., 1990]. As with the Loewe Additivity model there is no closed-form solution to this constraint equation, so the effect level satisfying this constraint must be determined at each C_{X}, C_{Y} using numerical root-finding techniques. And as with Loewe additivity, the Greco synergy model applies equally well to any quantitative measure of effect.

Potentiation: This model may be applied to cases where the Y compound directly increases or decreases the X compound's ability to inhibit the biological process. The inhibition for a potentiated model is:
I=X(C′_{X})
where C′_{X} is C_{X} (1+C_{Y}/C₀)^{π}.
Here, C₀ is the threshold Y concentration at which potentiation becomes important, and pi (π) is the potentiation index governing the degree of synergism produced. The Potentiation model applies in the specified form equally well to any quantitative measure of effect, and is not limited to inhibition measurements.

The four models may be used describe the inhibition surfaces that occur for a simple pathway containing a single branch. However, in an embodiment of the invention, the four models may also be used collectively to predict the behaviour of more complicated networks, since all networks structures are built up of fundamental connections that produce the same possible target relationships between paired inhibitors. Other embodiments of the invention may utilize a subset of the four models, or may utilze a set including other interaction models, to collectively predict the behavior of a network system.

The four models were constructed in the context of pairwise inhibitors of a branched pathway, but may be generalized to multiply inhibited systems with more than two perturbers. All of the models may be generalized to situations using more than two inhibitors.

The four models were constructed in the context of an inhibited enzymatic pathway, but can equally be modified to describe any perturbative effect on any kind of network, whether biological or not, as known to those skilled in the art. As well, other embodiments of the invention may utilize one or more of the four models modified to predict a related variable to inhibition (e.g., a measure of inhibition normalized on a background measurement), wherein the conversion between the related variable and inhibition is understood by those skilled in the art.

Thus, in an embodiment of the invention, a method of elucidating the connectivity in a network system that has been subjected to a plurality of agents, the agents having an interaction in the system, includes the steps of providing the set of four interaction models; selecting an interaction model from the set that best models the interaction of agents in the system; and relating the selected model to connectivity of the network.

This method may also be used to predict and test the effects of combined perturbers on specific networks, wherein the specific networks may be analogous to one or more other networks for which the interaction of perturbers on the other networks is known. Similarly, the method may be used to identify a potential mechanism of agents (e.g., therapeutic compounds) in network systems based on the identified connectivity in a known network system from the interaction of agents. The method may also be used to elucidate unexpected connections between agents that act in combination upon a system (for example diet and medication).

Alternatively, this method may provide constraints for constructing connectivity models from observed combination effects on networks of unknown structure, thus, for example, providing the required understanding to identify novel targets for therapeutic compounds. This method can similarly be used to optimize high throughput screening methods for therapeutic combinations, and to highlight subsets of the data that will produce enhanced probability of therapeutic effect.

Any method described above may have one or all of the processes implemented in a computer system. Such implementations may include a series of computer instructions fixed either on a tangible medium, such as a computer readable medium (e.g., a diskette, CD-ROM, ROM, or fixed disk) or transmittable to a computer system, via a modem or other interface device, such as a communications adapter connected to a network over a medium. The medium may be either a tangible medium (e.g., optical or analog communications lines) or a medium implemented with wireless techniques (e.g., microwave, infrared or other transmission techniques). The series of computer instructions embodies all or part of the functionality previously described herein. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (e.g., shrink wrapped software), preloaded with a computer system (e.g., on system ROM or fixed disk), or distributed from a server or electronic bulletin board over a network (e.g., the Internet or World Wide Web). Of course, some embodiments of the invention may be implemented as a combination of both software (e.g., a computer program product) and hardware. Still other embodiments of the invention are implemented as entirely hardware, or entirely software (e.g., a computer program product).

All aforementioned embodiments of the invention are intended to be merely exemplary and numerous variations and modifications will be apparent to those skilled in the art. All such variations and modifications are intended to be within the scope of the present invention as defined in the appended claims.

Additional information concerning embodiments of the invention herein is provided in the attached, unpublished document entitled “Probing Biological Systems with Drug Combinations” authored by J. Lehár, G. Zimmermann, L. Giusti, R. Molnar, M. Lee, G. Serbedzija, and C. Keith, which is hereby incorporated herein by reference (see Appendix A).

### EXAMPLE

The following example is provided to illustrate some embodiments of the invention, and is not intended to limit the scope of any particular embodiment utilized.

In an example, an experiment is conducted to correlate the four interaction models with the combination effects of compounds that target the sterol biosynthesis pathway in yeast, as well as to highlight which other antifungals are most likely to combine synergistically with sterol pathway inhibitors.

A series of compounds that inhibit the sterol pathway in fungi, as well as a wider collection of antifungal compounds grouped by their target networks, was selected. A fungal growth assay using C. glabrata strain 90031, on 384-well plates was then run. Each combination was arrayed into 36 wells, providing a 6×6 combination surface similar in format to the surface shown in FIG. 1. Fungal growth was determined using a metabolic assay (Alamar Blue).

Each matrix was then extracted from the plates using CombinatoRx's informatics platform [Borisy et al. 2003, Proc. Natl. Acad. Sci., USA, 100(13):7977-7982]. Along with each inhibition matrix, a corresponding error matrix was calculated, based on the well-to-well variability of the assay in untreated wells, and further increased by the standard deviation of neighboring wells within each matrix, to account for compound delivery irregularities.

Once the dose response and error matrices were collected, we analyzed each of them in the following manner:
(1) Each dose response matrix was compared to all four models by measuring the volumetric difference between the observed surface and the model surface measured from the observed single agent curves on each matrix. To calculate the additivity surface, we applied an iterative root-finding method to solve the Loewe additivity relation [similar to Pritchard & Shipman 1990, Antiviral Res 14:181-206]. The novel model surfaces were directly calculated using the observed single agent dose response curves. The volumetric difference was summed over the entire combination space for each matrix, and an error estimate was provided by summing the squares of the constituent well errors.
(2) The best fit model was then selected to be the case which had the smallest chi-quared goodness-of-fit measure, provided that the inhibition surface had a total volume exceeding the volumetric noise (to exclude failed elements where there was no significant inhibition for any combined concentrations).

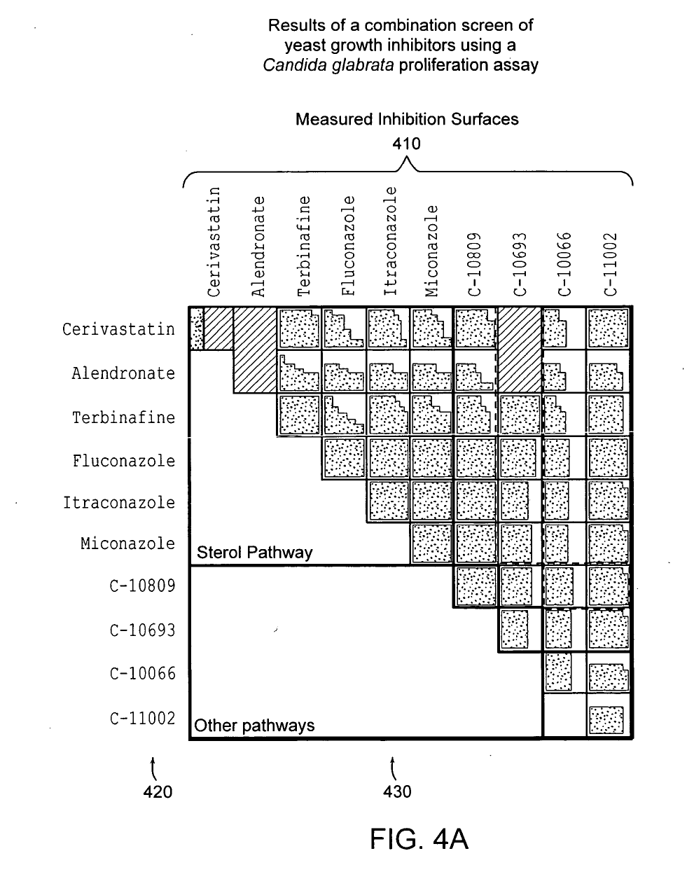

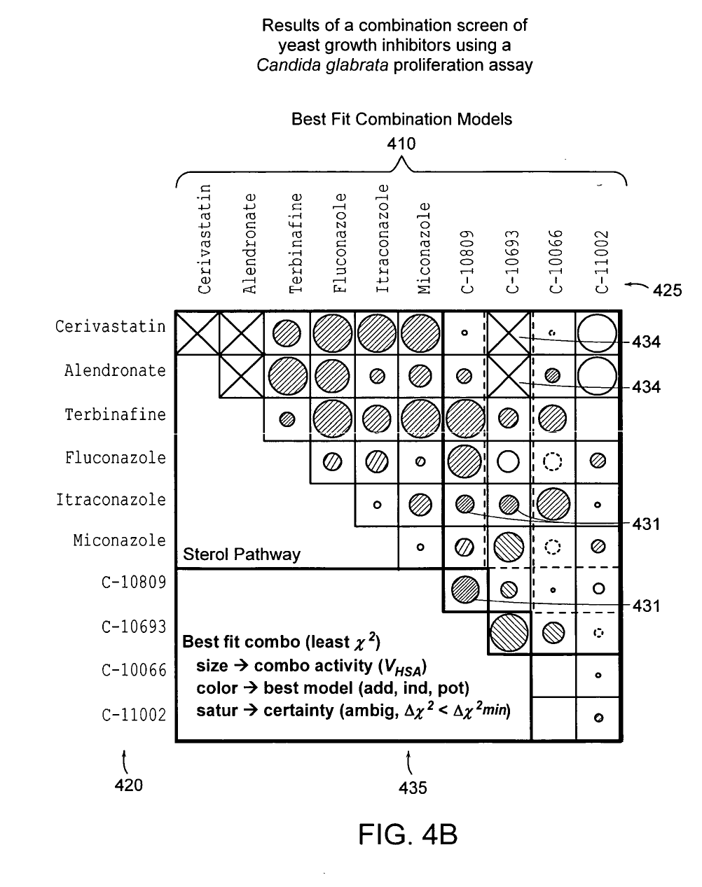

The resulting set of combination effects and combination effect calls is summarized by the representation presented in FIG. 4. The antifungal compounds in the experiment 410 are arranged along the axes 420, 425, and grouped according to their function. The left-hand matrix 430 shows the observed combination effect surfaces, similar to those in FIGS. 1-3. The circles in the right-hand matrix 435 show how strong a combination effect was observed (the strength correlating with the size of the circle) and which model was most closely fit (the model correlating with the color of the circle). The grey circles 431 are experiments in which the combination effect could not be unambiguously classified into one of the combination models, because two or more of the predicted model surfaces were similar enough that they could not be distinguished over the noise level from measurement errors. Boxes with an ‘X’ 434 represent combinations that were not tested. The reliability of the model determination can be gauged by those elements along the diagonal of the matrix, which should all fit either Loewe additivity or be grey (ambiguous model assignment).

Within the sterol pathway (comparing our results to the network structure summarized in Wills, Redinbo, Perfect, Del Poeta, 2000, Emerging Therapeutic Targets 4(3):1-32), compounds whose targets are known to be the same also tend to be Loewe additive, as expected (e.g., between the azoles). The inhibitors with targets within the sterol pathway clearly divide into those which share a target, producing predominantly Loewe additive effects, as expected, and those with different targets, producing Potentiation like effects (the analysis did not include Greco synergism models, but these would all have fit that model well, as expected for separate target combinations after generations of exponential cell proliferation). Combinations between compounds targeting different pathways show very different distributions of best fit models, supporting the notion that combination effects contain important target relationship information.

As a result of this experiment, a class of antifungal compounds that is especially synergistic with sterol pathway inhibitors may be identified, and characterization of the network relationship between the two pathways may be performed. Other compounds with similar properties may be tested as high-priority antifungal candidates in combination with sterol pathway inhibitors. Similar analyses may be applied to many assays to create prioritizations to permit more efficient combination screening.

1. A method of elucidating connectivity in a network system that has been subjected to a plurality of agents, the agents having an interaction in the system, the method comprising:
a. providing a set of interaction models for describing an interaction of agents in the system;
b. selecting an interaction model from the set that best models the interaction of agents in the system; and
c. relating the selected model to connectivity of the network.

2. A method according to claim 1, wherein the network system includes at least one of a chemical system, biochemical system, and biological system.

3. A method according to claim 1, wherein the plurality of agents includes at least one composition.

4. A method according to claim 3, wherein the composition includes a pharmaceutically active composition.

5. A method according to claim 3, wherein the composition includes an entity approved by a governmental regulatory agency for administration to a patient.

6. A method according to claim 3, wherein the composition includes an entity having at least one of an established safety profile, a recognized pharmacology profile, and a recognized toxicity profile.

7. A method of identifying an interacting agent having an interaction with a network system according to claim 1, the method further comprising:
d. identifying the interacting agent having the interaction in the network system based on the connectivity of the network.

8. A method according to claim 7, wherein the interacting agent is not one of the plurality of agents.

9. A method according to claim 7, wherein the interacting agent is at least part of a pharmaceutically active composition.

10. A pharmaceutically active composition comprising:
an interacting agent identified according to claim 9; and
another agent identified based on the interaction between the another agent and the interacting agent in the network.

11. A method of using a pharmaceutically active composition to produce an interaction in an organism comprising:
identifying an interacting agent according to claim 9;
combining the interacting agent with another agent, identified based on the interaction between the another agent and the interacting agent in the network system, to produce the pharmaceutically active composition; and
administering the pharmaceutically active composition to the organism, the organism having the network system.

12. A method according to claim 9, wherein the interacting agent includes an entity approved by a governmental regulatory agency for administration to a patient.

13. A method of identifying an interacting agent with an interaction in a particular network system according to claim 1, the method further comprising:
d. repeating steps a, b, and c for each of a plurality of network systems; and
e. identifying the interacting agent with the interaction in the particular network system based on the connectivity of at least one of the plurality of network systems.

14. A method according to claim 13, wherein the interacting agent is not one of the plurality of agents.

15. A method according to claim 13, wherein the particular network system is not one of the plurality of network systems.

16. A method according to claim 13, wherein the interacting agent is at least part of a pharmaceutically active composition.

17. A pharmaceutically active composition comprising:
an interacting agent identified according to claim 16; and
another agent identified based on the interaction between the another agent and the interacting agent in the particular network.

18. A method of using a pharmaceutically active composition to produce an interaction in an organism comprising:
identifying an interacting agent according to claim 16;
combining the interacting agent with another agent, identified based on the interaction between the another agent and the interacting agent in the particular network system, to produce the pharmaceutically active composition; and
administering the pharmaceutically active composition to the organism, the organism having the particular network system.

19. A method according to claim 13, wherein the interacting agent includes an entity approved by a governmental regulatory agency for administration to a patient.

20. A method of elucidating a potential mechanism of interaction of a particular composition according to claim 1, wherein the plurality of agents includes at least one composition, the method further comprising:
identifying the potential mechanism of interaction of the particular composition in a particular system based on the connectivity of the network.

21. A method according to claim 16, wherein the particular composition is not one of the at least one composition.

22. A method according to claim 16, wherein the particular composition includes an entity approved by a governmental regulatory agency for administration to a patient.

23. A method of elucidating connectivity in a network system that has been subjected to a plurality of agents, the method comprising:
a. providing a set of interaction models for describing an interaction of agents in the system;
b. determining an interaction of at least one of the plurality of agents in the system;
c. selecting an interaction model from the set that best models the interaction of agents; and
d. relating the selected model to connectivity of the network.

24. A method according to claim 23, wherein determining the interaction includes using a high throughput screening method.

25. A method according to claim 23, wherein the plurality of agents includes at least three agents, the method further comprising:
selecting at least one more interaction models from the set, each interaction model best models a particular interaction of agents in the system; and
relating each selected model to the connectivity of the network.

26. A method according to claim 1, wherein at least one of the interaction models is a Loewe additivity model.

27. A method according to claim 26, wherein the Loewe additivity model is represented by the constraint for an effect level I at combined concentration Cₓ,C_{Y}
$$\frac{{C}_{X}}{{EC}_{X}} + \frac{{C}_{Y}}{{EC}_{Y}} = 1$$
where C_{X}, C_{Y} are the concentrations of two agents for a particular combination treatment, and EC_{X}, EC_{Y} are the effective concentrations of the two agents individually.

28. A method according to claim 1, wherein at least one of the interaction models is an Independence model.

29. A method according to claim 28, wherein the Independence model is represented by
I=X+Y−XYγ
wherein I is the predicted inhibition of a combination of compositions X and Y at concentration C_{X} and C_{Y}, respectively; X is the single expected inhibition of a compound X at concentration C_{X}; Y is the single expected inhibition of a compound Y at concentration C_{Y}; gamma (γ) is the interaction parameter and describes the degree to which the single agents interact to produce a combination effect; and
wherein gamma may have the value represented by the expressions
γ=1;
γ=(X_{∞}+Y_{∞}−1)/(X_{∞}Y_{∞});
γ=1/max(X_{∞},Y_{∞});
γ=0;
γ=(X_{∞}+Y_{∞})/(X_{∞}Y_{∞}); or
any other value corresponding to a specific interaction of agents in the network system.

30. A method according to claim 1, wherein at least one of the interaction models is a Greco synergism model.

31. A method according to claim 30, wherein the Greco synergism model is represented by the constraint
$$\frac{{C}_{X}}{{EC}_{X}} + \frac{{C}_{Y}}{{EC}_{Y}} + α ( \frac{{C}_{X}}{{EC}_{X}} \frac{{C}_{Y}}{{EC}_{Y}} ) = 1$$
wherein I is the predicted inhibition of a combination of compositions X and Y at concentration C_{X} and C_{Y}, respectively; where C_{X}, C_{Y} are the concentrations of the two agents for a particular combination treatment, and EC_{X}, EC_{Y} are the effective concentrations of the single agents (the single agent concentrations that can produce the same level of effect as at the specified combination); and alpha (α) represents the strength of synergistic interaction and has values of −1 through infinity.

32. A method according to claim 1, wherein at least one of the interaction models is a Potentiation model.

33. A method according to claim 32, wherein the Potentiation model is represented by
I=X(C′X)
wherein I is the predicted inhibition of a combination of compositions X and Y at concentration C_{X} and C_{Y}, respectively; X is the single expected inhibition of a compound X at concentration C_{X}; Y is the single expected inhibition of a compound Y at concentration C_{Y}; and
where C′_{X} is C_{X}(1+C_{Y}/C₀)/^{π} and C₀ is the threshold Y concentration at which potentiation becomes important, and pi (π) is the potentiation index governing the degree of synergism produced.

34. A method according to claim 1, wherein selecting the interaction model includes selecting the interaction model based on a least squares method.

35. A method of preparing a high throughput screen according to claim 1, the method further comprising:
preparing a high throughput screen based on the connectivity of the network.

36. A computer program product for use on a computer system for elucidating connectivity in a network system from an interaction of agents, the computer readable program code including:
a. module for collecting data related to the interaction of agents;
b. program code for calculating a predicted interaction of agents in a system for each of a set of interaction models, each model representing a particular connectivity of the network; and
c. program code for selecting an interaction model that best models the interaction of agents based on the calculated predicted interaction of agents.

37. A computer program product according to claim 36, wherein at least one interaction model is a Loewe additivity model.

38. A computer program product according to claim 37, wherein the Loewe additivity model is represented by the constraint for an effect level I at combined concentration Cₓ,C_{Y}
$$\frac{{C}_{X}}{{EC}_{X}} + \frac{{C}_{Y}}{{EC}_{Y}} = 1$$
where C_{X}, C_{Y} are the concentrations of two agents for a particular combination treatment, and EC_{X}, EC_{Y} are the effective concentrations of the two agents individually.

39. A computer program product according to claim 36, wherein at least one interaction model is an Independence model.

40. A computer program product according to claim 39, wherein the Independence model is represented by
I=X+Y−XYγ
wherein I is the predicted inhibition of a combination of compositions X and Y at concentration C_{X} and C_{Y}, respectively; X is the single expected inhibition of a compound X at concentration C_{X}; Y is the single expected inhibition of a compound Y at concentration C_{Y}; gamma (γ) is the interaction parameter and describes the degree to which the single agents interact to produce a combination effect; and
wherein gamma may have the value represented by the expressions
γ=1;
γ=(X_{∞}+Y_{∞}−1)/(X_{∞}Y_{∞});
γ=1/max(X_{∞},Y_{∞});
γ=1/min(X_{∞},Y_{∞});
γ=0;
=(X_{∞}+Y_{∞})/(X_{∞}Y_{∞}); or
any other value corresponding to a specific interaction of agents in the network system.

41. A computer program product according to claim 36, wherein at least one interaction model is a Greco synergism model.

42. A computer program product according to claim 41, wherein the Greco synergism model is represented by
$$\frac{{C}_{X}}{{EC}_{X}} + \frac{{C}_{Y}}{{EC}_{Y}} + α ( \frac{{C}_{X}}{{EC}_{X}} \frac{{C}_{Y}}{{EC}_{Y}} ) = 1$$
wherein I is the predicted inhibition of a combination of compositions X and Y at concentration C_{X} and C_{Y} respectively; where C_{X}, C_{Y} are the concentrations of the two agents for a particular combination treatment, and EC_{X}, EC_{Y} are the “effective concentrations” of the single agents (the single agent concentrations that can produce the same level of effect as at the specified combination); and alpha (α) represents the strength of synergistic interaction and has values of −1 through infinity.

43. A computer program product according to claim 36, wherein at least one interaction model is a Potentiation model.

44. A computer program product according to claim 43, wherein the Potentiation model is represented by
I=X(C′_{X})
wherein I is the predicted inhibition of a combination of compositions X and Y at concentration C_{X} and C_{Y}, respectively; X is the single expected inhibition of a compound X at concentration C_{X}; Y is the single expected inhibition of a compound Y at concentration C_{Y}; and
where C′_{X} is C_{X}(1+C_{Y}/C₀)^{π} and C₀ is the threshold Y concentration at which potentiation becomes important, and pi (π) is the potentiation index governing the degree of synergism produced.

45. A computer program product according to claim 36, wherein the program code for selecting the interaction model includes program code implementing a least squares method.

46. A method of producing an interaction model to describe an interaction of agents in a network system for elucidating connectivity in the system, the method comprising:
a. simulating interaction of agents in the system to produce a response surface; and
b. producing the interaction model based on the response surface.
